(19) United States
(12) Patent Application Publication
(10) Pub. No.: US 2025/0221921 A1
(43) Pub. Date: Jul. 10, 2025

(54) VERSATILE SYNERGISTIC POLYSACCHARIDE COMPOSITION FOR COSMETIC FORMULATIONS

(71) Applicant: Barnet Products, LLC, Englewood Cliffs, NJ (US)

(72) Inventors: Rossana Lanza, Glen Rock, NJ (US); Vincent Domenick, New Paltz, NY (US); Michelle Miller Howard, Warren, NJ (US); Haoyu Wang, Livingston, NJ (US); Katiuska Matos Horta, Hackensack, NJ (US)

(21) Appl. No.: 19/010,341

(22) Filed: Jan. 6, 2025

Related U.S. Application Data
(60) Provisional application No. 63/617,486, filed on Jan. 4, 2024.

(51) Int. Cl. A61K 8/9789; A61K 8/04; A61K 8/73; A61K 8/9717; A61Q 19/00

(52) U.S. Cl. CPC A61K 8/9789; A61K 8/044; A61K 8/737; A61K 8/9717; A61Q 19/007; A61K 2800/48

(57) ABSTRACT

A composition that may be used, but is not limited to, in a range of cosmetic products, personal care products, health care products or topical products, in the form of a serum, lotion, cream, gel, a spray, a foam, a solid stick, a shampoo, a hair conditioner, a lacquer, a make-up or a sunscreen. The composition including three plant-derived hydrocolloids: Carrageenan; Guar extract; and Tamarind seed extract.

This application claims priority to U.S. Provisional Patent Application No. 63/617,486, filed Jan. 4, 2024, entitled “VERSATILE SYNERGISTIC POLYSACCHARIDE COMPOSITION FOR COSMETIC FORMULATIONS” and is hereby incorporated by reference herein.

The present invention provides a novel polysaccharide composition for use within cosmetic and personal care products, offering enhanced formulation adaptability and performance across diverse ingredients.

### BACKGROUND

In today's personal care and cosmetics raw material market landscape, formulators are inundated with countless new functional materials with increasingly complex mechanisms of action. Given the ever-increasing competition amongst cosmetic finished goods companies, formulators must rapidly choose raw materials that have broad compatibility, functionality, and marketing appeal to formulate products that consumers will choose to buy over competitor products. The break-neck speed at which formulators must operate leaves little time to test the compatibility and synergistic capabilities of new raw materials. This often presents a difficult choice for formulators: either experimenting with new and innovative raw materials with unknown compatibilities and risking potential failures in stability or making small tweaks to existing bases and enabling rapid development of stable yet traditional formulations. Unfortunately, the riskier option carries a much greater potential reward, as consumers are more drawn to the most innovative and distinct formulas, increasing the likelihood of driving sales volume. Therefore, a middle-ground between these two choices is to use material complexes with carefully balanced ratios that have the greatest versatility and synergy when used in combination with some of the most used ingredients in personal care and cosmetics finished goods.

It is known that Guar Gum is a natural non-ionic polysaccharide. Chemically, Guar Gum is an exo-polysaccharide composed of the sugar galactose and mannose. Individually, Guar Gum shows a clear low shear plateau on the flow curve and is strongly shear-thinning. It also shows synergistic behavior with polysaccharides such as xanthan gum, starch, and cellulose gum. However, when using weight percentages above 1.5-2.0%, gels and emulsions become much less flowable, and the pick-up decreases, making it so the product behaves more like a stiff gelatin. Guar Gum is not commonly used alone, but instead is frequently combined with a variety of other gums to achieve higher viscosities without negatively affecting the rheology/aesthetic of a formula. Moreover, finding a suitable ratio between Guar and other gums that allow for the best stability, rheology, and aesthetics can be very challenging for a formulator.

Tamarind Gum is a water-soluble, high-molecular-weight, and plant-derived polysaccharide which has a glucose backbone with xylose and galactose side chains. Tamarind Gum powder can help form more elastic gels and imparts a lower viscosity than Guar and Carrageenan Gum. Tamarind gum is known for the silky texture it imparts on formulas, but much like Guar Gum, when Tamarind Gum is used above 2.0% by weight or so, topical products will ball up on skin as the product is rubbed in. This phenomenon can be somewhat countered by adding polyols at higher levels. However, higher percentages of polyols will add stickiness to the formula, which is usually an undesirable property. In addition, if not combined with other gums, Tamarind Gum may continue to swell over longer periods of time, causing a formula that was within its specifications on day 1 to drift out of specification.

Carrageenan is a polysaccharide naturally derived from red seaweeds of the solieriaceae family. According to the number and location of sulfuric acid groups, carrageenan is generally divided into three types: Iota, lambda, and kappa carrageenan. Iota Carrageenan is an excellent thickener and gelling agent and is often used in formulas for its versatility across a range of pHs and concentrations of salts. However, when used at higher percentages (above 1.5% by weight), even small amounts of salts may drastically increase viscosity and cause instability in formulations containing Iota Carrageenan, so it is important to combine this gum with others.

The present invention was formulated to specifically avoid the drawbacks of each of these gums and maximize their benefits.

### SUMMARY OF THE INVENTION

The present invention provides a composition including three plant-derived hydrocolloids such as Carrageenan; Guar extract; and Tamarind seed extract. The composition may be used, but is not limited to, in a range of cosmetic products, /personal care products, health care products or topical products, in the form of a serum, lotion, cream, gel, a spray, a foam, a solid stick, a shampoo, a hair conditioner, a lacquer, a make-up or a sunscreen.

### DETAILED DESCRIPTION OF THE INVENTION

The present invention utilizes a middle ground approach combining three specially selected hydrocolloids at specific proportions that synergize with a wide variety of commonly used cosmetic ingredients to be able to provide versatile formats that yield a unique sensorial feel without the common drawback of unpredictable viscosities, inconsistent textures, piling, tackiness and stringiness. The specific ingredients in the present invention are Guar Gum, Tamarind Seed Gum, and a specific type of Carrageenan of the iota variety.

The present invention was formulated to specifically avoid the drawbacks of each of these gums and maximize the benefits. Even when used at the maximum recommended use level of 3.0% by weight, each component falls below the threshold percentage of each gum that would cause undesirable effects. The charts provided below in Tables 1-4 show the individual percentages of each gum when used at different percentages in an example formulation referred to as “Barthix”:

TABLE 1

| | Impact on Formula Properties at | |
|---|---|---|
| | 0.5% Use Level | |
| Ingredient | Property | Comments |
| Barthix | Clarity | Turbid |
| (Sodium Carrageenan: 0.075-0.125% | Viscosity | Slight Gelation |
| Guar Gum: 0.1-0.175% | Application | More body than |
| Tamarind Gum: 0.125-0.25%) | | water, silky |
| | Afterfeel | Silky |
| | Dispersibility | Quick/No Clumps |
| Sodium Carrageenan | Clarity | Clear |
| | Viscosity | Water Thin |
| | Application | Feels like water |
| | Afterfeel | Same as before |
| | | application |
| | Dispersibility | Moderate/Some |
| | | clumping |
| Cyamopsis Tetragonoloba (Guar) | Clarity | Very Turbid |
| Gum | Viscosity | Slight Gelation |
| | Application | Sticky/Slimey |
| | Afterfeel | Sticky |
| | Dispersibility | Moderate/Some |
| | | Clumping |
| Tamarindus Indica Seed Gum | Clarity | Turbid |
| | Viscosity | Water Thin |
| | Application | Feels like water |
| | Afterfeel | Silky |
| | Dispersibility | Quick/No Clumps |

TABLE 2

| | Impact on Formula Properties at | |
|---|---|---|
| | 1.0% Use Level | |
| Ingredient | Property | Comments |
| Barthix | Clarity | Turbid |
| (Sodium Carrageenan: 0.15-0.25% | Viscosity | Moderate Gelation |
| Guar Gum: 0.2-0.35% | Application | Increased body, |
| Tamarind Gum: 0.25-0.5%) | | silky, quick |
| | | absorption |
| | Afterfeel | Silky, Light |
| | Dispersibility | Quick/No Clumps |
| Sodium Carrageenan | Clarity | Clear |
| | Viscosity | Water Thin |
| | Application | Feels like water |
| | Afterfeel | Same as before |
| | | application |
| | Dispersibility | Moderate/Some |
| | | clumping |
| Cyamopsis Tetragonoloba (Guar) | Clarity | Very Turbid |
| Gum | Viscosity | Moderate Gelation |
| | Application | Sticky/Slimey |
| | Afterfeel | Heavy/Sticky |
| | Dispersibility | Moderate/Some |
| | | Clumping |
| Tamarindus Indica Seed Gum | Clarity | Turbid |
| | Viscosity | Water Thin |
| | Application | Silky |
| | Afterfeel | Silky |
| | Dispersibility | Quick/No Clumps |

TABLE 3

| | Impact on Formula Properties at | |
|---|---|---|
| | 2.0% Use Level | |
| Ingredient | Property | Comments |
| Barthix | Clarity | Very Turbid |
| (Sodium Carrageenan: 0.3-0.5% | Viscosity | High Gelation |
| Guar Gum: 0.4-0.7% | Application | Increased body, |
| Tamarind Gum: 0.5-1.0%) | | silky, quick |
| | | absorption |
| | Afterfeel | Silky, Light film |
| | | without pilling |
| | Dispersibility | Quick/No Clumps |
| Sodium Carrageenan | Clarity | Clear |
| | Viscosity | Slight Gelation |
| | Application | Sticky/Draggy, Long |
| | | Playtime |
| | Afterfeel | Heavy, Sticky |
| | Dispersibility | Moderate/Some |
| | | clumping |
| Cyamopsis Tetragonoloba | Clarity | Very Turbid |
| (Guar) Gum | Viscosity | Gelatin like |
| | Application | Very Sticky/Draggy |
| | Afterfeel | Pilling, slightly |
| | | irritating |
| | Dispersibility | Difficult/Increased |
| | | clumping |
| Tamarindus Indica Seed Gum | Clarity | Turbid |
| | Viscosity | Water Thin |
| | Application | Sticky/Draggy, Long |
| | | Playtime |
| | Afterfeel | Silky |
| | Dispersibility | Quick/No Clumps |

TABLE 4

| | Impact on Formula Properties at | |
|---|---|---|
| | 3.0% Use Level | |
| Ingredient | Property | Comments |
| Barthix | Clarity | Very Turbid |
| (Sodium Carrageenan: 0.45-0.75% | Viscosity | High Gelation |
| Guar Gum: 0.6-1.05% | Application | Increased body, |
| Tamarind Gum: 0.75-1.5%) | | silky, quick |
| | | absorption |
| | Afterfeel | Silky, Light film |
| | | without pilling |
| | Dispersibility | Quick/No Clumps |
| Sodium Carrageenan | Clarity | Turbid |
| | Viscosity | Slight Gelation |
| | Application | Sticky/Draggy, |
| | | Long Playtime |
| | Afterfeel | Heavy, Sticky |
| | Dispersibility | Difficult/Increased |
| | | clumping |
| Cyamopsis Tetragonoloba (Guar) | Clarity | Very Turbid |
| Gum | Viscosity | Too thick to process |
| | Application | N/A |
| | Afterfeel | N/A |
| | Dispersibility | N/A |
| Tamarindus Indica Seed Gum | Clarity | Turbid |
| | Viscosity | Slight Gelation |
| | Application | Sticky/Draggy, |
| | | Long Playtime |
| | Afterfeel | Draggy |
| | Dispersibility | Moderate/Some |
| | | Clumping |

Carrageenan used in the present invention is from red seaweed extract. The red seaweed extract may be in the form of Carrageenan powder. In one embodiment of the present invention, Sodium Carrageenan is used. It can be in iota, lambda or kappa grades in ranges of 15% to 25% of the total amount by weight of Sodium Carrageenan, Guar Gum and Tamarind Gum. The iota is the preferred option. Examples of this include Ricovis 8870, GENUVISCO® Carrageenan, GENUGEL® Carrageenan, Viscarin® GP 109 NF, SeaSpen® GP379, AEC Carrageenan powder, and Cargill Carrageenan.

The Guar extract used in an embodiment of the present invention is derived from Cyamopsis Tetragonoloba and used at ranges of 25% to 35% of the total amount by weight of Sodium Carrageenan, Guar Gum and Tamarind Gum. The Guar extract may be in the form of Guar gum powder and may be non-ionic. Examples of this are, but not limited to, Guar Gum Powder HV-101, Activesoft S, AEC Guar Gum, Aurist Care GHI, Jaguar S, OriStar Guar Gum, and Vidocrem.

Tamarind seed extract used in the present invention is derived from the endosperm or kernel of the seeds of a tamarind tree. The Tamarind seed extract may be in the form of tamarind gum powder. The Tamarindus Indica Seed Gum used in an embodiment of the present invention, can be used from 25% to 50% of the total amount by weight of Sodium Carrageenan, Guar Gum, and Tamarind Gum. Examples of this include, but are not limited to, GLYLOID 3S, Tamavisco, Xilogel, and Sarda Temcol.

The present invention provides a composition of three unique hydrocolloids which when used in specific proportions, as shown in an example shown in Table 5, can boost the viscosity, stability, and sensorial application of a wide range of cosmetic and personal care formulations. The three hydrocolloids are present above a threshold that when used individually would cause undesirable effects including viscosity, drift, instability, and unfavorable texture. The invention avoids the drawbacks of individual gums and maximizes the benefits including good dispersibility, consistent viscosity and desirable texture. The innovation stems from the invention's intricately balanced composition, enabling easy integration into diverse cosmetic formulations. This proprietary composition highlights its compatibility with an array of cosmetic ingredients, exemplified through interactions with a variety of common ingredients such as starches and Xanthan Gum, and its favorable viscosity in the presence of salts.

TABLE 5
Composition

| | MATERIAL |
|---|---|
| INCI | % |
| Sodium Carrageenan | 15-25% |
| Cyamopsis Tetragonoloba | 20-35% |
| (Guar) Gum | |
| Tamarindus Indica Seed Gum | 25-50% |

The invention exhibits unique synergies across a wide variety of commodity ingredients frequently used in cosmetic and personal care formulations. Specifically, mixtures of the present invention synergize with starches and certain polysaccharides, both of which can create undesirable textures and aesthetics when used in other contexts. These synergies allow formulators to use reduced quantities of these ingredients without compromising their beneficial attributes. By harnessing the invention's synergistic properties, formulations attain improved textural properties and performance while circumventing typical drawbacks associated with these ingredients. Compositions made with the invention have a luxurious skin feel that mitigates tack and has quick absorption into the skin that provides long lasting hydration properties. There is no stringy affect as there is with xanthan gum.

Furthermore, the inventive combination provides a versatile thickening, stabilizing, and suspending quality in a range of formulations. It can be used as a gelling or thickening agent for aqueous systems. It may also be used as a suspending agent, film former, lubricant, rheology modifier, silicone alternative for feel, dispersant or stabilizer. Its synergistic viscosity effect and distinctive viscosity behavior in the presence of salts sets it apart from traditional thickeners. Unlike conventional options that experience reduced viscosity with salt addition, the inventive composition provides increased viscosity, providing formulators with enhanced stability and textural control across formulations containing salts.

The core innovation lies in the invention's adaptability and transformative impact on cosmetic formulations. Its ability to synergize with various ingredients broadens formulation possibilities, ensuring superior-quality products with enhanced stability, texture, and overall performance.

The invention's novel features and advantages include, but are not limited to, immediate skin hydrating properties and sensorial texture. It also has an ease of processing compared to other natural gums.

The invention's versatility extends across a spectrum of cosmetic and personal care products, including lotions, creams, gels, sprays, foams, sticks, shampoos, conditioners, lacquers, make-up, sunscreens and/or serums. Formulators can harness its versatile properties to create high-performance formulations meeting consumer demands for innovative products offering superior sensory experiences.

Skin care formulations using the inventive composition at 1% in a skin care final formulation, demonstrate quicker absorption into skin when tested in the same formulation against market standard ingredients containing a complex blend of xanthan gum and phospholipid known to hydrate and protect skin barrier. It has proven to have improved sensorial feel with more absorption, freshness and cooling feeling to skin when tested in the same formulation against traditional xanthan gum and market standard ingredients containing a complex of xanthan gum and phospholipid known to hydrate and protect skin barrier. It has also proven to have a significant hydrating effect which lasts up to 24 hours after one single application. It has proven to have less tackiness and stickiness compared to traditional Xanthan gum in the same formulation. It has also proven to have improved sensorial texture resulting in better spread ability when compared to traditional Xanthan gum and a xanthan gum/phospholipid blend in the same formulation. It has also proven to have more slip and smoothness compared to traditional Xanthan gum in the same formulation.

The invention represents a revolutionary polysaccharide complex for the cosmetic and personal market, unlocking new horizons in formulation design. Its exceptional adaptability and synergy with diverse ingredients propel the cosmetic industry towards innovative formulations that exceed existing standards in performance and user experience.

### FINISHED FORMULA EXAMPLES

The following non limiting examples are provided for illustration purposes in order to facilitate a better understanding of the disclosure. The ingredients presented here may be obtained from varied suppliers and may be substituted with other ingredients sharing the same INCI name.

### Example 1

In this example formula, the inventive composition referred to as “BarThix™ is paired with Xanthan Gum, a very commonly used aqueous thickener. (Shown in Table 6). Xanthan Gum provides some viscosity boost on its own but is significantly amplified when combined with BarThix™. Both thickeners are pre-mixed with a polyol (Butylene Glycol) before being dispersed into the water phase. Moreover, the addition of BarThix™ bestows greater control over sensorial and rheological properties of formulations.

### Example 1 Procedure

1. In the main beaker/vessel, combine Phase A and mix until uniform.
2. Premix Phase B in a separate container until uniform.
3. Add Phase B to Phase A and homogenize/mix until uniform. A thick gel will form.

TABLE 6

| Aqueous Gel Example Formula | 1 |
|---|---|
| INCI | MATERIAL % |
| Phase A | — |
| Water | QS |
| Phenoxyethanol | 0.3-1.0% |
| Phase B | — |
| Butylene Glycol | 1.0%-5.0% |
| BarThix ™ | 0.5-2.0% |
| Xanthan Gum | 0.1-0.3% |

### Example 2

In this example formula, BarThix™ is paired with Hydroxypropyl Starch Phosphate. (Shown in Table 7). On its own this starch has both thickening and emulsification properties when used at medium to high levels (5% +). However, when combined with BarThix™ the percentage can be reduced significantly. This allows greater freedom to the formulator by freeing up a portion of the formula. In addition, the benefits of the starch are retained and the detriments (dry after feel and pilling on skin) are mitigated. These qualities are controlled easily by modifying the percentage of BarThix™.

### Example 2 Procedure

1. In the main beaker/vessel, combine Phase A and mix until uniform.
2. Premix Phase B in a separate container until uniform.
3. Add phase B to Phase A and homogenize/mix until uniform. A thick gel will form.
4. Combine Phase C in a separate beaker/vessel and mix until uniform.
5. At a constant rate, add Phase C to main beaker/vessel while homogenizing. Mix well.

TABLE 7

| O/W w/Starch Example Formula | 2 |
|---|---|
| INCI | MATERIAL % |
| Phase A | — |
| Water | QS |
| Phenoxyethanol | 0.3-1.0% |
| Phase B | — |
| Butylene Glycol | 1.0%-5.0% |
| Hydroxypropyl Starch Phosphate | 1.0-5.0% |
| BarThix ™ | 0.5-2.0% |
| Phase C | — |
| Polyglyceryl-10 Myristate | 0.5-2.0% |
| Coconut Alkanes | 5.0-10.0% |
| Caprylic/Capric Triglyceride | 5.0-10.0% |

### Example 3

In this example formula, BarThix™ is paired with Sodium Chloride. (Shown in Table 8). Under normal circumstances, Sodium Chloride can disrupt emulsions, causing separation of oil and water phases before the formula is completed. When BarThix™ is added to the formula, it interacts synergistically with the salt, increasing the viscosity and overall integrity of the emulsion. In addition, salts may increase the efficacy of the preservation system by preventing microbial growth. Hence, by adding BarThix™, the formulator is capable of adding ingredients that increase the overall robustness of their formulations.

### Example 3 Procedure

1. In the main beaker/vessel, combine Phase A and mix until uniform.
2. Premix Phase B in a separate container until uniform.
3. Add phase B to Phase A and homogenize/mix until uniform. Begin heating main beaker to 80-85C.
4. Combine Phase C in a separate beaker/vessel and heat to 80-85C while mixing.
5. At a constant rate, add Phase C to main beaker/vessel while homogenizing. Mix until uniform.
6. Cool Batch to 25C while sweep mixing.

TABLE 8

| Salt Synergy Example Formula | 3 |
|---|---|
| INCI | MATERIAL % |
| Phase A | — |
| Water | QS |
| Phenoxyethanol | 0.3-1.0% |
| Sodium Chloride | 0.1-1.0% |
| Phase B | — |
| Glycerin | 1.0-5.0% |
| BarThix ™ | 0.5-2.0% |
| Phase C | — |
| Squalane | 5.0-10.0% |
| C9-12 Alkane Coco- | 5.0-10.0% |
| Caprylate/Caprate | |
| Hydrogenated Lecithin | 0.5-2.0% |

In the foregoing specification, the invention has been described with reference to specific embodiments thereof. It will, however, be evident that various modifications and changes may be made thereto without departing from the broader spirit and scope of the invention. The specification is, accordingly, to be regarded in an illustrative rather than a restrictive sense.

1. A composition consisting of three plant-derived hydrocolloids, wherein the three plant- derived hydrocolloids are Carrageenan; Guar extract; and Tamarind seed extract.

2. The composition according to claim 1, wherein the three plant-derived hydrocolloids are polysaccharides or gums.

3. The composition according to claim 1, wherein the Carrageenan is derived from Red seaweed extract.

4. The composition according to claim 1, wherein the Guar extract is derived from Cyamopsis tetragonoloba.

5. The composition according to claim 1, wherein the Tamarind seed extract is derived from the endosperm, or a kernel of the seeds of a tamarind tree.

6. The composition according to claim 3 wherein the red seaweed extract is in the form of carrageenan powder.

7. The composition according to claim 4, wherein the guar extract is in the form of guar gum powder.

8. The composition according to claim 5, wherein the tamarind seed extract is in the form of tamarind gum powder.

9. The composition according to claim 6 wherein the carrageenan powder is sodium carrageenan.

10. The composition according to claim 9 wherein the sodium carrageenan is iota sodium carrageenan.

11. The composition according to claim 7 wherein the guar gum powder is non-ionic.

12. The composition according to claim 10 wherein the amount of iota sodium carrageenan in the composition ranges from 15% to 25% of the total weight of the composition.

13. The composition according to claim 7 wherein the amount of guar gum powder in the composition ranges from 20% to 35% of the total weight of the composition.

14. The composition according to claim 8 wherein the amount of tamarind gum powder in the composition ranges from 25% to 50% of the total weight of the composition.

15. The composition according to claim 1, wherein the gum of each of the three plant-derived hydrocolloids is present at a concentration above the threshold that when used individually would cause viscosity drift, instability, and unfavorable texture.

16. The composition according to claim 1, wherein the composition maximizes good dispersibility, consistent viscosity, and desirable texture.

17. The composition according to claim 1, wherein the composition provides excellent formulation adaptability and long-lasting hydration to skin.

18. The composition according to claim 1, wherein the composition provides versatile thickening, stabilizing, and suspending ability in a range of formulations, wherein the recommended use is 0.01% to 3.0% of the composition in a total formulation.

19. The composition according to claim 1, wherein the composition synergizes with a commonly used cosmetic ingredients, including enhanced stability and textural control across formulations.

20. The composition according to claim 1, wherein the composition is used as a suspending agent, film former, lubricant, rheology modifier, gelling agent, thickening agent, alternative to silicone for feel, dispersant or stabilizer.
